(19) United States
(12) Patent Application Publication
(10) Pub. No.: US 2017/0100274 A1
(43) Pub. Date: Apr. 13, 2017

(54) Drop Foot Assistive Device

(71) Applicant: Louis Haines, New York, NY (US)

(72) Inventor: Louis Haines, New York, NY (US)

(21) Appl. No.: 15/289,906

(22) Filed: Oct. 10, 2016

Related U.S. Application Data
(60) Provisional application No. 62/239,421, filed on Oct. 9, 2015.

(51) Int. Cl. A61F 5/01

(52) U.S. Cl. CPC A61F 5/0113

(57) ABSTRACT

A drop foot assistive device for stabilizing a drop foot condition of a user is provided. The user has a leg, a foot, and toes with the foot encased in a shoe and the shoe having shoelaces. The drop foot assistive device comprises a shin wrap with a securing mechanism releasably securing the shin wrap around the leg. A drop foot band having a first end and a second end is connected to the shin wrap. The second end of the drop foot band is positioned between the shoe and the shoelaces and connected to the shoelaces closest to the toes such that during a walking motion of the user, the drop foot band creates a pulling action on the toes of the user, stabilizing the foot and achieving a natural walking motion.

### CLAIM OF PRIORITY

This patent application claims priority under 35 USC 119 (e) (1) from U.S. Provisional Patent Application Ser. No. 62/239,421 filed Oct. 9, 2015, of common inventorship herewith entitled, “Drop Foot Remedy,” which is incorporated herein by reference as though the same were set forth in its entirety.

### FIELD OF THE INVENTION

The present invention pertains to the field of therapeutic aids, and more specifically to the field of assistive apparati for drop foot.

### BACKGROUND OF THE INVENTION

With today's heightened awareness of health concerns and constantly improving medical care, humans are living longer than ever before. If unprecedented increase in life expectancy has a downside, it is exposure of risk to chronic age related disorders. Such serious ailments as diabetes, Alzheimer's and Parkinson's diseases are but a few of the disabling disorders that prevent many older persons from enjoying their longevity. Elderly persons also deal with physical infirmities brought on by inevitability of aging.

Suffering a stroke is a common occurrence that may result in limited mobility issues. Even what was once a natural endeavor like walking now is challenging for those who have suffered a stroke and a resulting symptom known as drop foot syndrome. Drop foot is a gait abnormality in which the dropping of the forefoot happens due to weakness, damage to the peroneal nerve or paralysis of the muscles in the anterior portion of the lower leg. Drop foot is characterized by an inability to dorsiflex or point one's toes toward the body or to move the foot at the ankle inward or outward. While most commonly caused by stroke, drop foot also effects people who had hip replacement surgery or suffer from the infirmities of old age as well as serious conditions such as ALS or Lou Gehrig's Disease, muscular dystrophy, multiple sclerosis, or cerebral palsy.

The prior art has put forth several designs of assistive apparati for drop foot. Among these are:

U.S. Pat. No. 8,382,694 to Jerry Wenger describes an ankle foot orthotic that treats steppage gate by preventing foot drop during movement. The orthotic having an ankle brace linked to a user's shoe by an elastic strap. The elastic strap having sufficient elasticity to provide the appropriate amount of tensile force to the shoe to prevent foot drop, while having sufficient flexibility to allow the user to naturally flex their foot and ankle during movement. The elastic strap comprises hook fasteners attachable to and removable from the shoe, such that the orthotic can be easily put on or taken off by the user.

U.S. Pat. No. 8,353,807 to Lambertus Joseph Martinus Kruijsen and Gunter Gneiding describes a method for compensating functional disabilities of the foot of a wearer including the steps of placing a shoe on a wearer and attaching a first attachment member about a lower leg of the wearer. The first attachment member has a first connecting member flexibly extending from the first attachment member and directed toward the foot. A second attachment member is attached in engagement with a shoe placed over the wearer's foot such that the second attachment has a second attachment member flexibly extending therefrom and directed toward the lower leg of the wearer. The first and second connecting members are coupled such that the flexible connecting member extends at an acute angle from the lower leg of the wearer at a point above the joint of a leg and foot to a point forward of the leg at the instep of the foot.

U.S. Pat. No. D514225 to Darlene Sassi describes an ornamental design for a drop foot aid that includes a calf mounted support strap, a vertical support strap, an ankle tie in strap, and a ring.

None of these prior art references describe the present invention.

### SUMMARY OF THE INVENTION

It is an object of the present invention to provide an assistive and therapeutic apparatus particularly configured to be worn on one's shin to help stabilize their foot and achieve a more natural walking gait.

The present invention is a drop foot assistive device for stabilizing a drop foot condition of a user. The user has a leg, a foot, and toes with the foot encased in a shoe and the shoe having shoelaces. The drop foot assistive device comprises a shin wrap having a top edge, a bottom edge opposite the top edge, a first edge, and a second edge opposite the first edge, an inner surface, and an outer surface opposite the inner surface. A securing mechanism releasably secures the shin wrap around the leg. A drop foot band having a first end and a second end is connected to the shin wrap. The second end of the drop foot band is positioned between the shoe and the shoelaces and connected to the shoelaces closest to the toes such that during a walking motion of the user, the drop foot band creates a pulling action on the toes of the user, stabilizing the foot and achieving a natural walking motion.

A method for stabilizing a drop foot condition of a user. The user has a leg, a foot, and toes with the foot encased in a shoe and the shoe having shoelaces. The method comprises providing a shin wrap having a top edge, a bottom edge opposite the top edge, a first edge, and a second edge opposite the first edge, an inner surface, and an outer surface opposite the inner surface, providing a drop foot band having a first end and a second end, connecting the first end of the drop foot band to the shin wrap, releasably securing the shin wrap around the leg, positioning the second end of the drop foot band between the shoe and the shoelaces, connecting the second end of the drop foot band to the shoelaces closest to the toes, initiating a walking motion of the user, creating a pulling action on the toes of the user, stabilizing the foot, and achieving a natural walking motion.

The present invention further includes a drop foot assistive device for stabilizing a drop foot condition of a user. The user has a leg, a foot, and toes with the foot encased in a shoe and the shoe having shoelaces. The drop foot assistive device comprises a shin wrap having a top edge, a bottom edge opposite the top edge, a first edge, and a second edge opposite the first edge, an inner surface, and an outer surface opposite the inner surface. At least one adjustable hook and loop fastening strap is mounted to the outer surface of the shin wrap for releasably securing the shin wrap around the leg. An adjustable drop foot band having a first end and a second end is connected to the shin wrap. An eyelet is secured to the second end of the drop foot band with the eyelet securable to the shoelaces. The second end of the drop foot band is positioned between the shoe and the shoelaces and the eyelet is connected to the shoelaces closest to the toes such that during a walking motion of the user, the drop foot band creates a pulling action on the toes of the user, stabilizing the foot and achieving a natural walking motion.

### BRIEF DESCRIPTION OF THE DRAWINGS

FIG. 1 is a perspective view illustrating a drop foot assistive device, constructed in accordance with the present invention, showing a shin wrap, a hook and loop fastening strap, a tension compensator for drop foot, and a bottom component for adjusting tension.

FIG. 2 is another perspective view illustrating the drop foot assistive device of FIG. 1, constructed in accordance with the present invention, with the device placed on an individual's left calf and foot, just below the left knee.

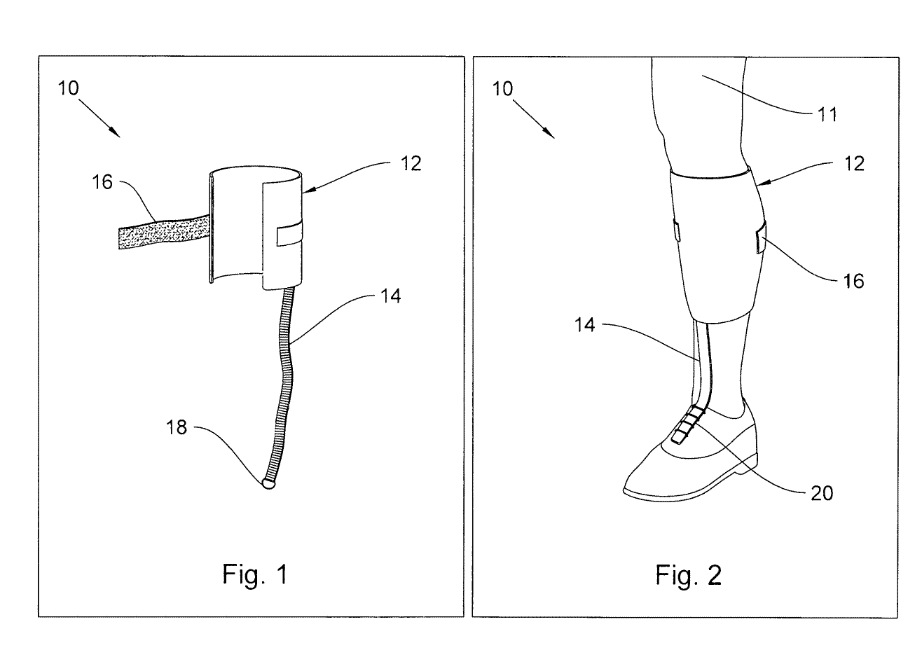

### DETAILED DESCRIPTION OF THE INVENTION

The present invention, hereinafter referred to as a Drop Foot Assistive Device, indicated generally at 10, is an assistive and therapeutic apparatus particularly configured to be worn on the leg 11 (i.e., the shin) to help stabilize the foot and achieve a more natural walking gait. The Drop Foot Assistive Device 10 provides those individuals who have suffered a stroke or other mobility impairment with a helpful aid in recovering walking ability.

The Drop Foot Assistive Device 10 of the present invention is comprised of basically two components, a shin wrap 12 and an integrated drop foot band 14 extending from the shin wrap 12. The shin wrap 12 has a top edge, a bottom edge substantially opposite the top edge, a first edge, and a second edge substantially opposite the first edge, an inner surface, and an outer surface opposite the inner surface. The shin wrap 12 preferably measures approximately five (5″) inches in width although having a shin wrap 12 with a greater width or lesser width is within the scope of the present invention.

The shin wrap 12 of the Drop Foot Assistive Device 10 of the present invention is positioned with the inner surface of the shin wrap 12 against the effected leg 11 and is wrapped around the effected leg 11 with the first edge of the shin wrap 12 overlapping the second edge of the shin wrap 12. At least one adjustable hook and loop fastening strap 16 is mounted to the outer surface of the shin wrap 12 and secures the shin wrap 12 around the effected leg 11 and accommodate any leg 11 size. In another embodiment, the Drop Foot Assistive Device 10 has a series of adjustable hook and loop fastening straps 14.

In addition, the Drop Foot Assistive Device 10 of the present invention includes the drop foot band 14 having a first end and a second end. The first end of the drop foot band 14 is secured to or adjacent to the bottom edge of the shin wrap 12 and extends downward from the bottom edge of the shin wrap 12. Preferably, the drop foot band 14 is a length of adjustable elastic banding that measures approximately two (2″) inches in width and has a metal or ABS plastic eyelet 18 secured to the second end of the drop foot band 14. The second end of the drop foot band 14 is preferably fed through the user's shoelaces 20 from the top of the foot to the toes and connects to a user's shoelace 20 at the shoelace's tip closest to the toes. Once the Drop Foot Assistive Device 10 is applied to the shin 11 and connected to the shoelace 20, the walking motion of the user creates a pulling action, the force of which is dependent on the set tension of the elastic drop foot band 14. For both non-elastic and elastic drop foot bands 14, the tension of the drop foot band 14 can also be adjusted by, connecting the second end of the drop foot band 14 at either different places on the shoe or wrapping the drop foot band 14 multiple times about the shoelaces, for instance. The set tension of the drop foot band 14 compensates for the drop foot by helping to replicate the motion of lifting one's leg while walking.

Comfortable and sturdy, the Drop Foot Assistive Device 10 of the present invention is donned in a matter of minutes to assist these users in improving their motor skills, returning them to a normal gait in a fraction of the time normally accorded to those afflicted with drop foot. In addition to reducing rehabilitation time, the Drop Foot Assistive Device 10 exercises a wearer's calf and leg muscles, strengthening them with each step taken. The Drop Foot Assistive Device 10 is discreetly covered with clothing. The Drop Foot Assistive Device 10 is highly functional for anyone with mobility challenges from sufferers of stroke, palsy and scoliosis to those individuals recovering from temporary disabilities stemming from surgery or broken bones. The Drop Foot Assistive Device 10 enables users to return to completing many everyday tasks in a pain free and unencumbered manner. Ideal for the home, and home health sector, the Drop Foot Assistive Device 10 is also a welcome addition to physical and occupational therapy facilities. Durably constructed of high quality materials, the Drop Foot Assistive Device 10 will withstand many years of continued use.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A drop foot assistive device for stabilizing a drop foot condition of a user, the user having a leg, a foot, and toes, the foot encased in a shoe, the shoe having shoelaces, the drop foot assistive device comprising:
a shin wrap having a top edge, a bottom edge opposite the top edge, a first edge, and a second edge opposite the first edge, an inner surface, and an outer surface opposite the inner surface;
securing means for releasably securing the shin wrap around the leg; and
a drop foot band having a first end and a second end, the first end of the drop foot band connected to the shin wrap;
wherein the second end of the drop foot band is positioned between the shoe and the shoelaces and connected to the shoelaces closest to the toes; and
wherein during a walking motion of the user, the drop foot band creates a pulling action on the toes of the user, stabilizing the foot and achieving a natural walking motion.

2. The drop foot assistive device of claim 1 wherein the inner surface of the shin wrap is positioned against the leg.

3. The drop foot assistive device of claim 2 wherein the shin wrap is wrapped around the leg with the first edge of the shin wrap overlapping the second edge of the shin wrap.

4. The drop foot assistive device of claim 1 wherein the securing means is at least one adjustable hook and loop fastening strap mounted to the outer surface of the shin wrap.

5. The drop foot assistive device of claim 1 wherein the first end of the drop foot band is secured to or adjacent to the bottom edge of the shin wrap.

6. The drop foot assistive device of claim 5 wherein the drop foot band extends downward from the bottom edge of the shin wrap.

7. The drop foot assistive device of claim 1 wherein tension of the drop foot band is adjustable.

8. The drop foot assistive device of claim 7 wherein the drop foot band is constructed from an elastic material.

9. The drop foot assistive device of claim 7 and further comprising:
an eyelet secured to the second end of the drop foot band, the eyelet securable to the shoelaces.

10. The drop foot assistive device of claim 9 wherein the eyelet is constructed from a metal material or an ABS plastic material.

11. A method for stabilizing a drop foot condition of a user, the user having a leg, a foot, and toes, the foot encased in a shoe, the shoe having shoelaces, the method comprising:
providing a shin wrap having a top edge, a bottom edge opposite the top edge, a first edge, and a second edge opposite the first edge, an inner surface, and an outer surface opposite the inner surface;
providing a drop foot band having a first end and a second end;
connecting the first end of the drop foot band to the shin wrap;
releasably securing the shin wrap around the leg;
positioning the second end of the drop foot band between the shoe and the shoelaces;
connecting the second end of the drop foot band to the shoelaces closest to the toes;
initiating a walking motion of the user;
creating a pulling action on the toes of the user;
stabilizing the foot; and
achieving a natural walking motion.

12. A drop foot assistive device for stabilizing a drop foot condition of a user, the user having a leg, a foot, and toes, the foot encased in a shoe, the shoe having shoelaces, the drop foot assistive device comprising:
a shin wrap having a top edge, a bottom edge opposite the top edge, a first edge, and a second edge opposite the first edge, an inner surface, and an outer surface opposite the inner surface;
at least one adjustable hook and loop fastening strap mounted to the outer surface of the shin wrap for releasably securing the shin wrap around the leg;
an adjustable drop foot band having a first end and a second end, the first end of the drop foot band connected to the shin wrap; and
an eyelet secured to the second end of the drop foot band, the eyelet securable to the shoelaces;
wherein the second end of the drop foot band is positioned between the shoe and the shoelaces and the eyelet is connected to the shoelaces closest to the toes;
wherein during a walking motion of the user, the drop foot band creates a pulling action on the toes of the user, stabilizing the foot and achieving a natural walking motion.

13. The drop foot assistive device of claim 12 wherein the inner surface of the shin wrap is positioned against the leg.

14. The drop foot assistive device of claim 13 wherein the shin wrap is wrapped around the leg with the first edge of the shin wrap overlapping the second edge wrap.

15. The drop foot assistive device of claim 12 wherein the first end of the drop foot band is secured to or adjacent to the bottom edge of the shin wrap.

16. The drop foot assistive device of claim 15 wherein the drop foot band extends downward from the bottom edge of the shin wrap.

17. The drop foot assistive device of claim 12 wherein the drop foot band is constructed from an elastic material.

18. The drop foot assistive device of claim 12 wherein the eyelet is constructed from a metal material or an ABS plastic material.
